(19) United States
(12) Plant Patent Application Publication
(10) Pub. No.: US 2016/0150696 P1
(43) Pub. Date: May 26, 2016

(54) Apple tree named 'Luresweet'

(50) ;

(71) Applicant: Lubera AG, Buchs (CH)

(72) Inventor: Markus Kobelt, Buchs (CH)

(21) Appl. No.: 14/544,112

(22) Filed: Nov. 25, 2014

(51) Int. Cl. A01H 5/00

(52) U.S. Cl. PLT/161

(57) ABSTRACT

A new and distinct Malus domestica plant that produces apples having a red flesh color and good eating quality.

### BOTANICAL CLASSIFICATION

Malus domestica

### VARIETAL DENOMINATION

‘Luresweet’

### BACKGROUND OF THE INVENTION

The present invention comprises a new and distinct cultivar of apple tree botanically classified as Malus domestica and known by the varietal name ‘Luresweet’. The new variety was discovered in October of 2006 in Buchs, Switzerland. The new variety is the result of planned breeding program, in which Malus domestica ‘LubA688’ (female parent, unpatented) was crossed with Malus domestica ‘LubA275’ (male parent, unpatented). The purpose of the breeding program was to develop red-fleshed apples having an improved eating quality. The new variety exhibits similar sugar content, size, and scab resistance to its female parent, but has a higher acidity content and red flesh. The new variety exhibits similar red flesh and level of acidity to its male parent, but has a better eating quality. The red flesh color combined with good eating quality of ‘Luresweet’ distinguishes the new variety from other Malus domestica varieties. Its red flesh color, high sugar and acidity content, and scab resistance are similar to Malus domestica ‘Lurechild’ (U.S. Plant Patent concurrently applied-for herewith). However, ‘Luresweet’ has a later harvest time, as well as taller, bigger, and more firm fruit, than ‘Lurechild’.

The new variety has been trial and field tested and has been found to retain its distinctive characteristics and remain true to type through successive propagations.

### DESCRIPTION OF THE DRAWINGS

The accompanying photographic drawings illustrate the new cultivar, with the color being as nearly true as is possible with color illustrations of this type:

FIG. 1 is a close-up view of a fruit of the new variety at maturity on the tree;

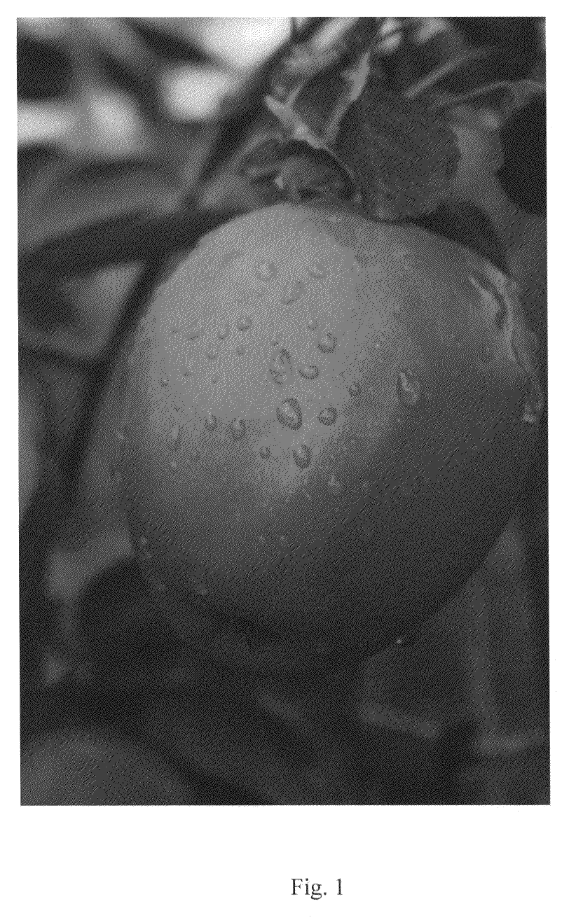

FIG. 2 shows fruits of the new variety at maturity on the tree;

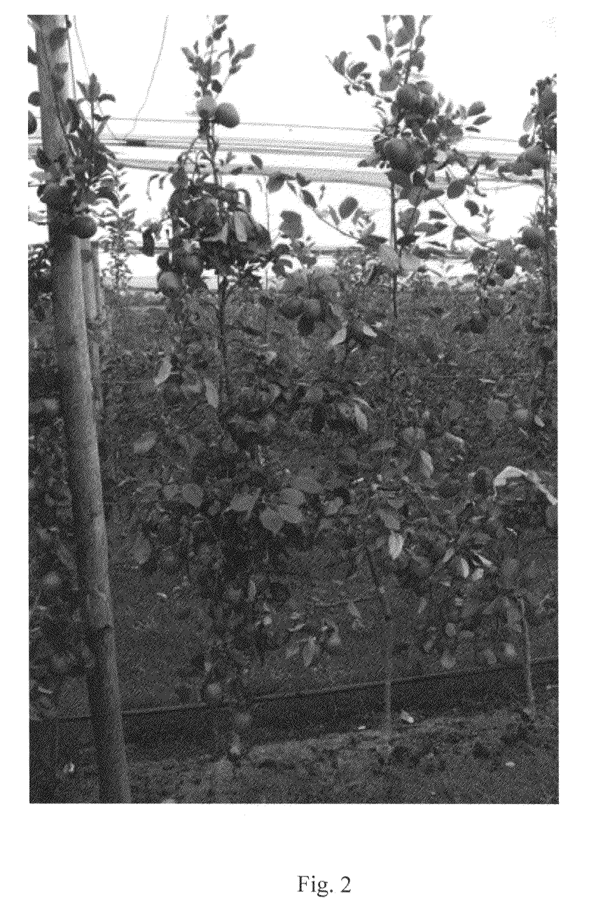

FIG. 3 is a longitudinal, cross-sectional view of a fruit of the new variety;

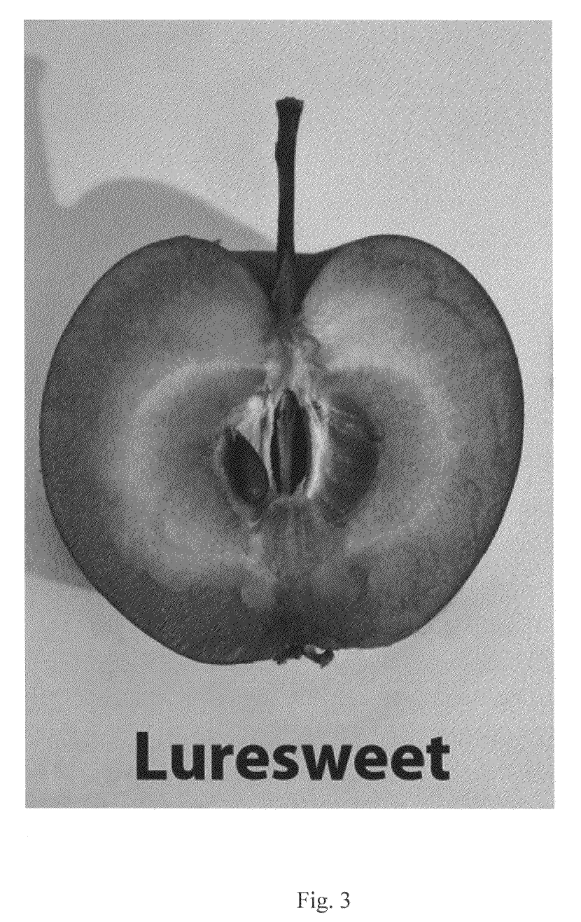

FIG. 4 is transverse, cross-sectional view of a fruit of the new variety;

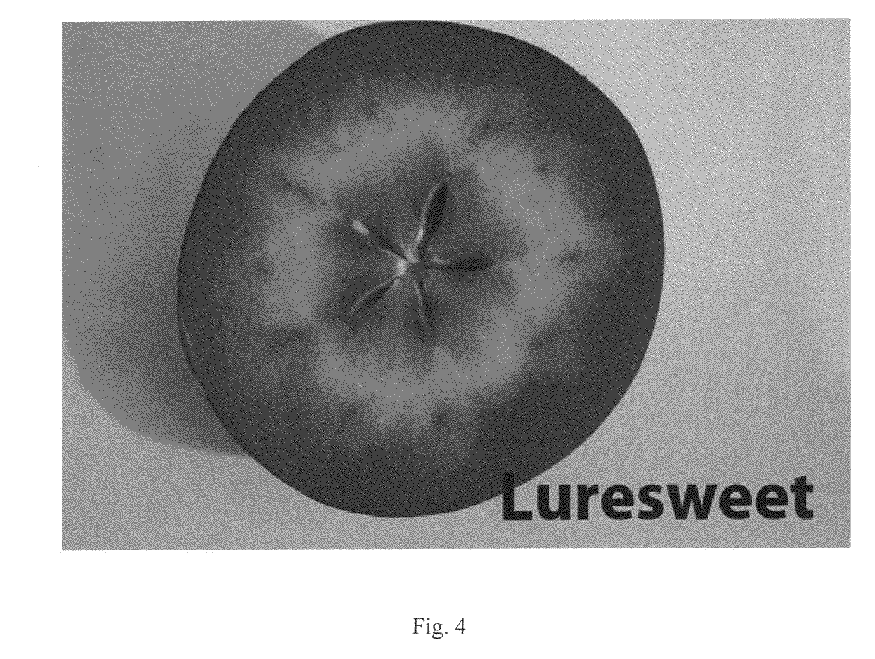

FIG. 5 shows buds and flowers of the new variety;

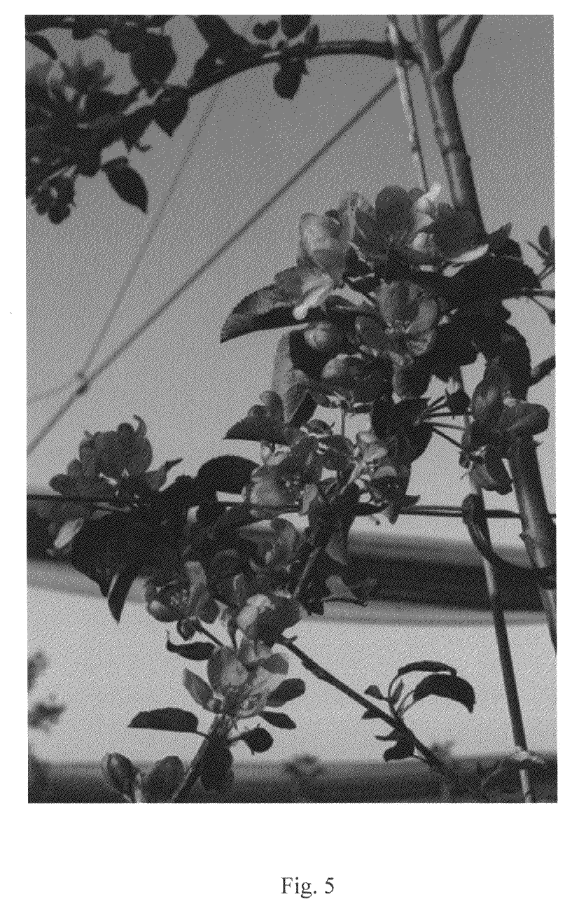

FIG. 6 is a close-up view of the flowers of the new variety;

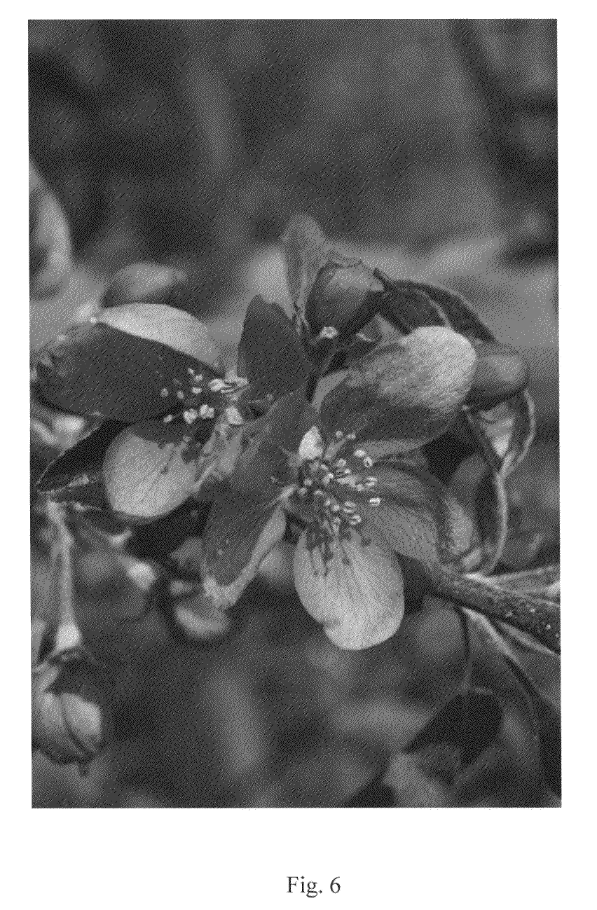

FIG. 7 shows upper and lower surfaces of young leaves of the new variety; and

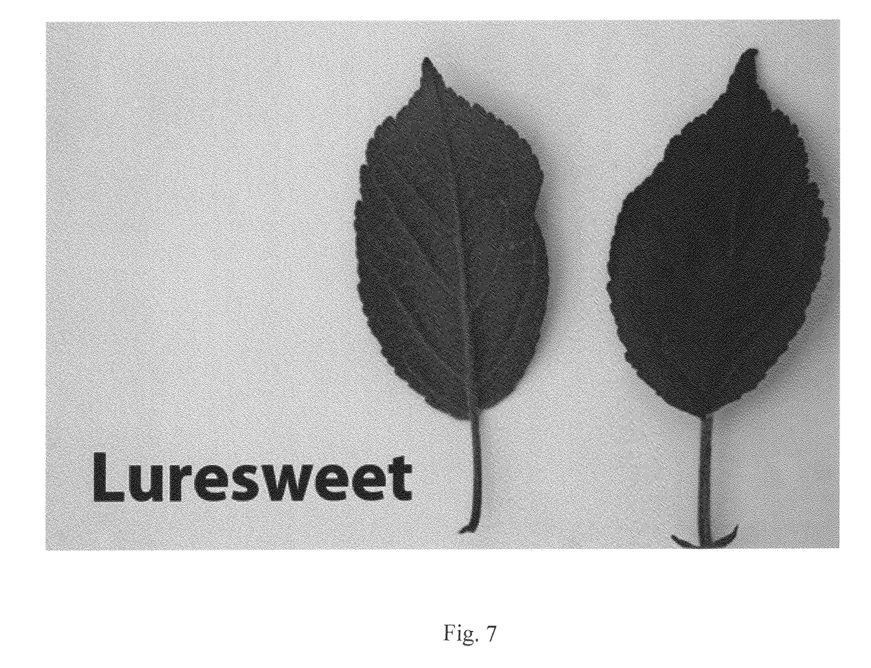

FIG. 8 shows upper and lower surfaces of mature leaves of the new variety.

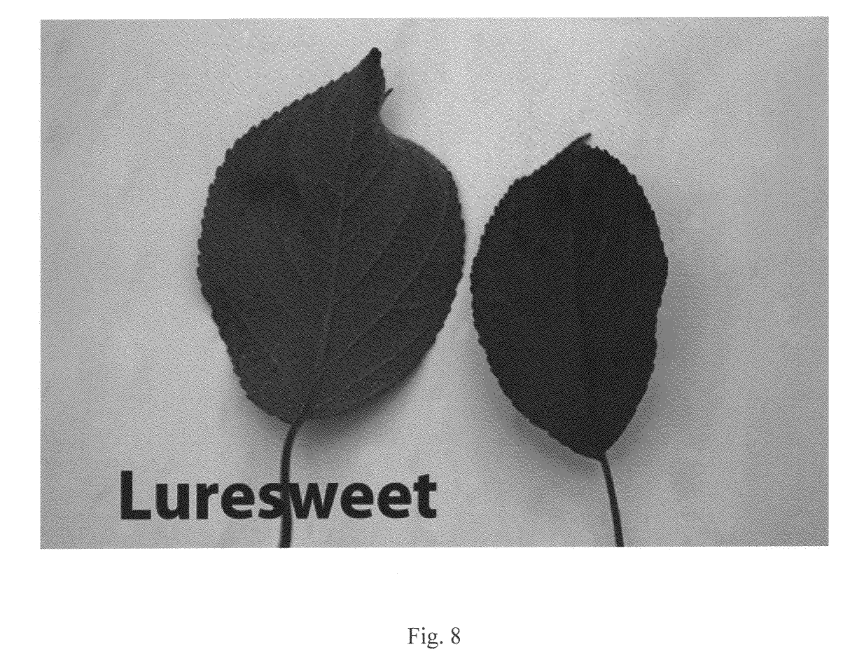

### DESCRIPTION OF THE PLANT

The following detailed description sets forth the characteristics of the new cultivar. The data which defines these characteristics was collected by asexual reproductions carried out via grafting on Malus domestica rootstocks in the Spring of 2007 in Buchs, Switzerland. The new variety was grown in apple orchard under standard apple tree conditions in Buchs, Switzerland. The color readings were taken in natural daylight without direct sunlight. Color references are primarily to the 2007 R.H.S. Colour Chart of The Royal Horticultural Society of London.
Tree:
   Age.—4 years.
   Size.—2.2×0.5 m.
   Spread.—3.2×0.5 m.
   Production.—7 kg per tree.
   Vigor.—Medium.
   Growth type.—Ramified; spreading.
   Trunk.—Size: Average of 18.0 mm in diameter. Surface texture: Uneven. Bark color: 176B. Lenticels: Length: 2.5 mm. Width: 1.0 mm. Color: 156A. Density: High.
   Branches.—Diameter: Average of 6.0 mm. Surface texture: Smooth, with pubescence at the distal half of shoots. Average angle: 40-50°. Lenticels: Length: 2.0 mm. Width: 1.0 mm. Shape: Oval to round. Density: High.
   Leaves.—Length: 8.0-10.0 cm. Width: 6.5-8.0 cm. Shape: Oval to rounded. Base: Large and round. Texture: Thick and leathery with a slight shine. Pubescence: Upper surface: Absent. Lower surface: Weak. Color: Young leaves: Upper surface: 183A. Lower surface: 178A to 178B. Mature leaves: Upper surface: N139A. Lower surface: 147B. Petiole: Shape, when viewed in cross-section: Slightly crenate. Length: 3.5-4.0 cm. Diameter: 1.5-1.8 mm. Color: 184B.
   Flowers.—Buds: Pedicels: Length: 21.0 mm. Diameter: 2.0 mm. Color: 187C. Bud (terminal): Length: 8.0 mm. Width: 4.0 mm. Color: 59A. Blooming time: The middle to the end of April in Buchs, Switzerland. Blooming period: Approximately 15 days. Pollination requirements: Cross-pollination is necessary.
   Fruit.—Date of picking: Beginning of October (similar to or just after ‘Golden Delicious’). Size: Axial diameter: 70.0-80.0 mm. Transverse diameter: 66.0-76.0 mm. Form: Conical to ovoid. Cavity: Regular in shape. Depth: 10.0 mm. Width: 20.0 mm. Basin: Ribbed. Depth: 8.0 mm. Width: 18.0 mm. Calyx: Mostly closed, with a small opening.
   Skin.—Thickness: Medium. Texture: Smooth. Color: 46A. Ground Color: 1D.
   Flesh.—Color: 48B. Texture: Very firm. Eating quality: Very good; high sugar and acidity; aromatic.
   Core.—Bundle area: Meeting at the core. Calyx tube: V-shaped; 10.0 mm from tube to shoulder. Styles: Generally closed, but a little open. Stamens: Median. Seed cells: Wall: Open. Depth: 18.0 mm. Breadth: 6.0 mm. Longitudinal section: 16.0-17.0 mm.
   Seeds.—Number perfect: 10. Number in one cell: 2. Length: 9.0-10.0 mm. Breadth: 6.0 mm. Form: Elongated and pointed. Color: 166A.
   Stem.—Length: 20.0-22.0 mm. Width: 2.0 mm. Color: 165A.
   Use.—Table or dessert fruit with good storage capability.
   Shipping quality.—No bruising with normal handling.
   Keeping quality.—˜3 months in a normal, cool storage area, but very sensitive to storage temperatures below 4-5° C.
   Tree winter hardiness.—Hardy to −18° C.
   Bud winter hardiness.—Hardy to −18° C.
   Drought tolerance.—Average.
   Disease resistance.—Resistant to scab.

I claim:

1. A new and distinct variety of Malus domestica tree substantially as shown and described.
